(19) United States
(12) Patent Application Publication
(10) Pub. No.: US 2022/0000943 A1
(43) Pub. Date: Jan. 6, 2022

(54) COMPOSITION COMPRISING TETRAGENOCOCCUS HALOPHILUS FOR PREVETION OR TREATMENT OF BEHCET'S DISEASE OR HERPES SIMPLEX VIRUS INFECTION

(71) Applicant: AJOU UNIVERSITY INDUSTRY-ACADEMIC COOPERATION FOUNDATION, Suwon-si (KR)

(72) Inventor: Seong Hyang SOHN, Suwon-si (KR)

(73) Assignee: AJOU UNIVERSITY INDUSTRY-ACADEMIC COOPERATION FOUNDATION, Suwon-si (KR)

(21) Appl. No.: 17/281,673

(22) PCT Filed: Oct. 1, 2019
(86) PCT No.: PCT/KR2019/012829

§ 371 Date: Mar. 31, 2021

(30) Foreign Application Priority Data
Oct. 8, 2018 (KR) 10-2018-0119827

(51) Int. Cl. A61K 35/744; A61P 37/06; A23L 33/00; A23L 33/135; A23L 33/125

(52) U.S. Cl. CPC A61K 35/744; A61P 37/06; A23V 2002/00; A23L 33/135; A23L 33/125; A23L 33/40

(57) ABSTRACT

A pharmaceutical composition or a health functional food composition for preventing or treating Behcet's disease have Tetragenococcus halophilus, a culture thereof or a lysate thereof. In addition, a pharmaceutical composition or a health functional food composition for preventing or treating a herpes simplex virus infection have Tetragenococcus halophilus, a culture thereof or a lysate thereof. The composition may further include a prebiotic compound. The Tetragenococcus halophilus produces lactate, and the lactate can be converted into short-chain fatty acids such as butyrate in the body, can control the expression of activating molecules of dendritic cells, and can effectively prevent or treat Behcet's disease or herpes simplex virus infection.

### TECHNICAL FIELD

The present invention relates to a composition for preventing or treating Behcet's disease or herpes simplex virus infection comprising Tetragenococcus halophilus.

### BACKGROUND ART

Behcet's disease is a rare and refractory inflammatory disease with symptoms of recurrent aphthous stomatitis of the oral and/or genital organs, uveitis, and skin lesions. Clinical symptoms include not only skin ulcers, but also multifaceted severe chronic inflammation of joints, central nervous system, gastrointestinal, kidney, genitourinary, lung, cardiovascular, intestinal bleeding, and digestive system related symptoms such as intestinal perforation, upper and lower venous syndrome, and aortic reflux. These symptoms are associated with systemic vasculitis and are a central pathophysiological feature of Behcet's disease.

Herpes simplex virus infection is an infection caused by Herpes virus hominis, and its symptoms are similar to those of Behcet's disease. There are skin diseases such as herpes simplex, eczema herpeticum and traumatic herpes; mucosal diseases such as aphthous stomatitis, recurrent stomatitis, acute herpetic rhinitis and genital herpes; and eye diseases such as conjunctivitis, keratoconjunctivitis and corneal ulcer.

Although the exact cause of Behcet's disease has not been identified, it is related to the autoimmune and autoinflammatory reactions triggered by herpes simplex virus infection. It is also used as an animal model for Behcet's disease by infecting the herpes virus into the ear auricle of mice, causing symptoms of Behcet's disease.

In Behcet's disease, macrophages and dendritic cells, as well as CD4+ T cells, CD8+ T cells, NK cells, neutrophils, and the like are involved, thereby invading cells. It is also associated with an increase in cytokine production. As the expression of microbiome in Behcet's disease patients is different from that of normal people, the immune response to some viruses and bacteria can be related to the inflammatory response of Behcet's disease.

Behcet's disease may show only mild symptoms such as skin mucosal inflammation or arthritis, or it can be accompanied by severe uveitis or serious sequelae by invading major organs such as the brain, lungs, heart and kidneys. Herpes simplex virus infection also has a problem that may cause disorders due to complications in the brain and eyes.

### DISCLOSURE

### Technical Problem

In order to solve the above problems, the present invention provides a pharmaceutical composition for preventing or treating Behcet's disease or herpes simplex virus infection comprising Tetragenococcus halophilus.

The present invention provides a health functional food composition for preventing or improving Behcet's disease or herpes simplex virus infection comprising Tetragenococcus halophilus.

### Technical Solution

The pharmaceutical composition for preventing or treating Behcet's disease according to the present invention may comprise Tetragenococcus halophilus, a culture thereof or a lysate thereof.

The health functional food composition for preventing or improving Behcet's disease according to the present invention may comprise Tetragenococcus halophilus, a culture thereof or a lysate thereof.

The pharmaceutical composition for preventing or treating a herpes simplex virus infection according to the present invention may comprise Tetragenococcus halophilus, a culture thereof or a lysate thereof.

The health functional food composition for preventing or improving herpes simplex virus infection according to the present invention may comprise Tetragenococcus halophilus, a culture thereof or a lysate thereof.

### Advantageous Effects

A pharmaceutical composition for preventing or treating Behcet's disease or herpes simplex virus infection comprising Tetragenococcus halophilus according to the present invention, can prevent rare intractable Behcet's disease or herpes simplex virus infection and can effectively treat, if an onset occurs. The therapeutic effect may be further increased by using the pharmaceutical composition in combination with existing therapeutic agents for Behcet's disease, inflammatory diseases caused by Behcet's disease, and herpes simplex virus infection.

In addition, it is possible to safely prevent and improve Behcet's disease or herpes simplex virus infection by various foods containing a health functional food composition for preventing or improving Behcet's disease or herpes simplex virus infection comprising Tetragenococcus halophilus according to the present invention.

### DESCRIPTION OF DRAWINGS

FIG. 1 shows the results according to an experimental example of the present invention.

### BEST MODE

Hereinafter, the present invention will be described in more detail.

The present invention provides a pharmaceutical composition for preventing or treating Behcet's disease, or a pharmaceutical composition for preventing or treating herpes simplex virus infection, comprising Tetragenococcus halophilus, a culture thereof or a lysate thereof.

In addition, the present invention provides a health functional food composition for preventing or improving Behcet's disease, or a health functional food composition for preventing or improving herpes simplex virus infection, including Tetragenococcus halophilus, a culture thereof, or a lysate thereof.

As used herein, “prevention” refers to any action of inhibiting or delaying the onset of Behcet's disease, herpes simplex virus infection, or at least one symptom thereof by administrating the pharmaceutical composition or health functional food composition according to the present invention. In addition, it includes treatment of a subject with remission of the disease for the prophylaxis and the prevention of recurrence.

As used herein, “treatment” refers to any action that improves or beneficially alters the condition, such as alleviating, reducing, or eliminating the onset of Behcet's disease, herpes simplex virus infection, or at least one symptom thereof by administrating the pharmaceutical composition according to the present invention.

As used herein, “improvement” refers to any action of that improves or beneficially alters the condition, such as alleviating, reducing, or eliminating the onset of Behcet's disease, herpes simplex virus infection, or at least one symptom thereof by ingestion of the health functional food composition according to the present invention.

As used herein, “pharmaceutical composition” means a composition administered for a specific purpose, and for the purposes of the present invention, it refers to be administered to prevent or treat Behcet's disease, herpes simplex virus infection, or at least one symptom thereof.

As used herein, “health functional food” has a meaning similar to food for specified health use (FoSHU), and refers to foods with high medical and medicinal effects processed so that the biological regulation function is effectively shown in addition to nutritional supply.

The pharmaceutical composition and health functional food composition for preventing or treating Behcet's disease or herpes simplex virus infection according to the present invention may comprise Tetragenococcus halophilus.

As used herein, “Tetragenococcus halophilus” is a halophilic lactic acid bacterium (LAB) found in fermentation processes for soy sauce, miso, fish sauce, and salted anchovies, etc., which is also known as “Pediococcus halophilus”. Tetragenococcus halophilus is gram-positive cocci of 0.6 to 1 μm, has no mobility and does not form spores.

The Tetragenococcus halophilus is vegetable lactic acid bacteria, and it can withstand acidic digestive juices of the stomach or small intestine better than that of animal lactic acid bacteria that live in animal foods such as milk and yogurt, and thus it has the effect of promoting intestinal activity, enhancing immunity and removing harmful bacteria in the intestine similar to those of animal lactic acid bacteria, and has the vitality of at least 400 times stronger. In addition, vegetable lactic acid bacteria have an ability to remove aflatoxin, a toxin from fungi, at least eight times higher than that of animal lactic acid bacteria.

In general, the genus of Tetragenococcus is typical lactic acid bacteria (LAB) of Gram-positive, catalase-negative, and oxidase-negative. Lactic acid bacteria decompose sugars such as glucose into lactic acid by metabolism. Physiologically, Tetragenococcus is distinguished from other lactic acid bacteria by its high salt resistance and its ability to grow at high pH values. The genus Tetragenococcus consists of a limited number of species including T. koreensis, T. muriaticus, T. osmosphilus, and T. solitarius, as well as Tetragenococcus halophilus.

The composition according to the present invention may contain only a single species of Tetragenococcus halophilus. The composition may not contain any other bacterial genera or species other than the Tetragenococcus halophilus. When the composition contains at least one other bacterial genus or species other than the Tetragenococcus halophilus, the other bacterial genus or species may be included only in minimal amount or a biologically irrelevant amount.

The composition according to the present invention may further contain other materials other than the Tetragenococcus halophilus. The composition may comprise one or more other bacterial genera or species. The composition may contain one or more other bacterial genera or species capable of living symbiotically in vivo with the Tetragenococcus halophilus.

The composition according to the present invention may comprise a culture of Tetragenococcus halophilus, or a lysate thereof. The culture may include a culture solution itself cultured in a medium, or a processed product derived from the culture solution itself such as a filtrate obtained by filtering or centrifuging the culture solution to remove a strain. The lysate may include a lysate obtained by crushing the strain and a supernatant obtained by centrifuging the lysate. In addition, the composition may comprise a mixture of live, i.e., active Tetragenococcus halophilus and killed Tetragenococcus halophilus.

The composition according to the present invention can be used for the prevention or the treatment of Behcet's disease. Behcet's disease (BD) is a rare and intractable chronic inflammatory disease that can affect various organs such as skin, blood vessels, gastrointestinal tract, central nervous system, heart and lungs, as well as oral ulcers, genital ulcers and eye inflammation. The targets to be prevented, improved or treated by the present invention are not limited to Behcet's disease. They may also include other inflammatory diseases associated with or caused by Behcet's disease.

In addition, the composition according to the present invention can be used for the prevention or the treatment of herpes simplex virus infection or inflammatory diseases caused by it. Herpes simplex virus infection is a viral disease caused by infection with the Herpes simplex virus, and herpes simplex virus is also called a herpes simplex virus, and when infected with it, various symptoms including mild symptoms such as blisters on the skin mucosa to severe diseases such as encephalitis appear. Like shingles virus, once infected, the herpes simplex virus exists in a person's body for a lifetime and since it is usually dormant, symptoms do not appear, but symptoms may recur if the virus is reactivated by stimulation.

The cause of Behcet's disease is not yet clearly known, but it has been known for a long time that in patients with a genetic predisposition, the immune response is activated by the addition of environmental factors such as herpes simplex virus infection, and as a result, various symptoms appear. Therefore, when suppressing the excessive immune response and simultaneously reducing the inflammatory response, symptoms of Behcet's disease or herpes simplex virus infection can be prevented, ameliorated or treated.

In the composition according to the present invention, the Tetragenococcus halophilus may decompose saccharides to produce organic acids. In an analysis experiment of microbial abundance, bacterial colonies and metabolites by fermentation through a soybean paste sample, when lactate, acetate, etc. increase rapidly by decrease of glucose and fructose, it was found that Tetragenococcus is the most dominant in the microbial population of the sample, and thus that Tetragenococcus is mainly involved in the production of organic acids.

The Tetragenococcus halophilus can degrade saccharides to produce the lactate, and the lactate can be converted into short-chain fatty acids (SCFAs) such as acetate and butyrate by intestinal microbes in the body. That is, the lactate produced by Tetragenococcus halophilus may be a precursor for the synthesis into butyrate.

The collection of all microbial groups that naturally exist in the human body is called a human microbiome, and the collection of genes of the human intestinal microbial community is called an intestinal microbiome. From birth, human intestinal microbes have a diverse community structure for each individual according to heredity, diet and lifestyle.

When comparing the intestinal microbial structure and the production of short-chain fatty acids (SCFAs) between Behcet's disease patients and healthy ordinary people through the intestinal microbiome, the differences in the intestinal microbial community can be found. The Behcet's disease patients showed relatively significantly reduced levels of Roseburia, Subdoligranulum, and the like compared to the healthy general population. In general, healthy people have a lot of probiotics (beneficial bacteria) such as Roseburia and Subdoligranulum which play an important role in gastrointestinal homeostasis, but Behcet's disease patients have a unique intestinal microbial imbalance. Accordingly, it was confirmed that the production of short-chain fatty acids such as butyrate was significantly reduced in the Behcet's disease patient.

Among human intestinal environment, especially the large intestine, is a bioreactor in which anaerobic microorganisms up to 10¹¹ cfu/g live and polysaccharides that are difficult to digest by humans such as plant-derived pectin, cellulose, hemicellulose and enzyme resistant starch are decomposed by the microorganism. In this process, short-chain fatty acids such as lactate, acetate and butyrate are produced by fermentation.

Among them, butyrate is the final product of the fermentation process carried out only by anaerobic bacteria, and increases intestinal motility in proportion to the production amount thereof, and can regulate metabolic activity and proliferation of cells as an important energy source for intestinal epithelial cells. In addition, the butyrate has beneficial functions for health such as suppressing the occurrence of cancer, strengthening the immune system, reducing inflammation and suppressing oxidative stress. For example, the butyrate may suppress the activation of the transcription factor NF-κB, thereby reducing the production of proinflammatory cytokines, resulting in an anti-inflammatory effect. In addition, depending on the concentration of the butyrate, cell growth may be inhibited or differentiation may be promoted, thereby inducing apoptosis in tumor cells, and acting as a nutrient factor for cells of intact tissue.

In addition, the butyrate can promote the differentiation of regulatory T cells. The regulatory T cells (Treg) include both natural regulatory T cells and adaptive regulatory T cells in vivo, and the butyrate promotes or enhances all mechanisms of regulatory T cells and it acts as an immune control so that the immune response in the body maintains a normal state. That is, it means that the immune suppression response is promoted or enhanced.

In the case of Behcet's disease patients, regulatory T cell responses may be reduced due to defects in the production of butyrate by a decrease in intestinal bacteria such as Roseburia and Subdoligranulum, and the activation of immune-pathological T cell responses may be induced. Accordingly, autoimmune diseases such as Behcet's disease may occur.

In the composition according to the present invention, the Tetragenococcus halophilus can control the expression of an activating molecule in dendritic cells. The Tetragenococcus halophilus has been shown to modulate the expression of activating molecules of dendritic cells, such as CD83 and CD40, in normal mouse experiments.

In the present invention, “dendritic cells (DC)” are immune cells consisting of the mammalian immune system, which presents in tissues in contact with the external environment such as the skin (a specialized form of dendritic cells called Langerhans cells) and the nose, the interface of the inside of lungs and intestines and can act as a professional antigen presenting cell. Their main function is to process antigenic substances and express them on the cell surface to present them to T cells. As a result, they act as messengers that connect the innate and adaptive immune systems.

Innate immunity is a major driver of non-specific immune activation in response to foreign substances. Immature dendritic cells are distributed through surrounding tissues that specialize in antigen internalization and allow continuous antigen monitoring, and are regulated by various signals from infectious and foreign substances, which trigger the differentiation and maturation of dendritic cells.

Molecules that begin to be expressed upon dendritic cell differentiation and activation help link innate immunity and acquired immunity. For example, CD83 is a cell surface marker that is primarily expressed on mature dendritic cells, and is strongly expressed along with co-stimulatory molecules such as CD80 and CD86, which provide co-stimulatory signals necessary for T cell activation and initiation of antigen-specific immune responses during the maturation of dendritic cells. CD83 is involved in the regulation of antigen presentation and can modulate the activation of dendritic cells and immune responses. CD40 is a costimulatory membrane glycoprotein found in antigen-presenting cells and mediates a wide range of immune and inflammatory responses, including T cell-dependent immunoglobulin class switching, memory B cell development, and the like. Ligation of CD40 promotes T cell activation by enhancing the expression of co-stimulatory molecules and inducing the release of IL-12. The differentiated T cells then organize a complex interaction of acquired immune responses.

In addition, dendritic cells induce differentiation into regulatory T cells that can inhibit the function of activated T cells, thereby playing an important role in the prevention of autoimmunity and transplant rejection of transplanted tissues and the induction of immune tolerance (self-tolerance) to autogenous antigens.

However, when there is a problem in inducing or maintaining self-tolerance, an immune response to the self-antigen occurs, causing a phenomenon that attacks the own tissue, and the disease caused by this process is called an autoimmune disease. The Behcet's disease may be an example of the autoimmune disease, and in addition, rheumatoid arthritis, type 1 diabetes (insulin dependent), systemic lupus, Crohn's disease, psoriasis, and the like are included in autoimmune diseases.

The composition according to the invention may further comprise at least one suitable prebiotic compound.

In the present invention, the “prebiotic compound” is a non-digestible component that increases the growth of specific microorganisms in the gastrointestinal tract and becomes a nutrient source of probiotics (beneficial bacteria) to activate probiotics or increase the number of strains. The prebiotic compound is resistant to hydrolysis and digestion and absorption by enzymes such as gastric acid in mammals, is fermented by the action of the probiotics, and can be a substance that selectively stimulates the proliferation and activity of the probiotics.

The prebiotic compounds are generally non-digestible carbohydrates such as oligo-, polysaccharides or sugar alcohols, and may be at least one selected from the group consisting of fructo-oligosaccharides (FOS), isomalto-oligosaccharides (IMO), xylo-oligosaccharide (XOS), chito-oligosaccharide (COS), pectin, inulin, β-glucan, polydextrose, D-tagatose and acacia fibers.

The amount of the prebiotic compound and its activity characteristics may vary depending on the sort, type and combination of the probiotics. For example, the composition may comprise the prebiotic compound in an amount of about 1 to about 30% by weight, preferably 5 to 20% by weight, based on 100 weight of the total composition, and the activity of the Tetragenococcus halophilus may be changed by the prebiotic compound and the amount of butyrate finally produced may also vary.

In the composition according to the present invention, each of the pharmaceutical compositions may be formulated and used in various forms according to conventional methods. The pharmaceutical composition may be formulated so that it can be rapidly or continuously delivered into the body. For example, it may be formulated as an oral dosage form of capsule, tablet, granule, powder, suspension or syrup. In addition, the Tetragenococcus halophilus may be provided as the pharmaceutical composition in freeze-dried, spray-dried or viable forms.

The oral formulations may include an enteric formulation. The enteric formulation includes an enteric coating so that it has gastric resistance and the pharmaceutical composition can be suitably delivered to the intestine. For example, a pharmaceutical composition according to the present invention can be designed to release its contents when it reaches the location of the intestine having a pH greater than about 6.8. In addition, the oral formulation may be essentially formed into an enteric formulation without the enteric coating.

The capsule agent may include a soft capsule. The soft capsule may have a certain elasticity and flexibility due to the addition of a softening agent present in the capsule shell, for example, glycerol, sorbitol, maltitol and polyethylene glycol. The soft capsule may be formed based on gelatin or starch, and may have various shapes, for example, cylindrical, oval, rectangular or torpedo forms.

The pharmaceutical composition according to the present invention can be formulated and used in a parenteral dosage form. The parenteral dosage form may include sterile injection solutions and suppositories.

The pharmaceutical composition according to the present invention can be prepared by additionally including a pharmaceutically acceptable carrier, excipient, or diluent according to the formulation. The “pharmaceutically acceptable” means exhibiting properties that are not toxic to cells or humans exposed to the pharmaceutical composition.

Carriers, excipients or diluents that can be used in the present invention include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate or mineral oil and the like.

When the pharmaceutical composition according to the present invention is formulated in the above form, it may be prepared using diluents or excipients such as fillers, weighting agents, binders, wetting agents, disintegrants and surfactants that are commonly used. For example, when Tetragenococcus halophilus according to the present invention is included in the pharmaceutical composition as a single bacterium, it may be formulated to contain one or more other bacterial strains and fillers in order to maintain the stability of the bacterium.

Solid preparations for oral administration include tablets, pills, powders, granules, capsules, and the like, and such a solid preparation may be prepared by mixing at least one excipient, for example, starch, calcium carbonate, sucrose or lactose, etc.

Further, in addition to simple excipients, lubricants such as magnesium stearate and talc may also be used. Liquid preparations for oral use include suspensions, liquid solutions, emulsions, syrups, etc. and in addition to water and liquid paraffin, which are commonly used simple diluents, various excipients such as wetting agents, sweetening agents, fragrances and preservatives may be included.

The pharmaceutical composition according to the invention may be particularly effective when used in combination with an additional therapeutic agent. The pharmaceutical composition may further include a compound generally used for the treatment of Behcet's disease or herpes simplex virus infection, such as antibiotics, anti-inflammatory agents, anesthetics, analgesics, and the like. This may vary slightly according to each formulation, but it is not limited thereto.

In the composition according to the present invention, the pharmaceutical composition may be administered in a pharmaceutically effective amount. The “pharmaceutically effective amount” refers to an amount sufficient to treat a disease at a reasonable benefit/risk ratio applicable to medical treatment and does not cause side effects, and the effective dose level can be determined according to factors including the patient's health status, type of ulcer, severity, activity of the drug, sensitivity to the drug, method of administration, time of administration, route of administration and rate of excretion, duration of treatment, drugs used in combination or concurrently, and other factor well known in the medical field.

For example, a suitable daily dose of bacteria for an adult human may be about 1×10³ to about 1×10¹¹ CFU (colony forming unit), e.g., about 1×10⁷ to about 1×10¹⁰ CFU, for another example about 1×10⁶ to about 1×10¹⁰ CFU.

The composition according to the present invention may contain a bacterial strain in an amount of about 1×10⁶ to about 1×10¹¹ CFU/g based on the weight of the composition. For example, it may be about 1×10⁷ to about 1×10¹⁰ CFU/g. The dosage can be, for example, 1 g, 3 g, 5 g and 10 g.

The pharmaceutical composition according to the present invention may be administered individually or in combination with other therapeutic agents, and may be administered sequentially or simultaneously with conventional therapeutic agents. It can also be administered singly or multiple times. Considering all of the above factors, it is important to administer an amount capable of obtaining the maximum effect in a minimum amount without side effects.

In the composition according to the present invention, the health functional food composition may also be prepared in various forms such as capsules, tablets, granules, powders, suspensions or syrups.

The health functional food composition may contain various nutrients, vitamins, minerals (electrolytes), flavors such as synthetic flavors and natural flavors, etc., colorants and fillers (cheese, chocolate etc.), pectic acid and its salts, alginic acid and its salts, organic acids, protective colloid thickeners, pH adjusting agents, stabilizers, preservatives, glycerin, alcohols, carbonating agents used in carbonated drinks, and the like. It may also contain flesh for the production of natural fruit juices, synthetic fruit juices and vegetable drinks. These components may be used independently or in combination.

The health functional food may be in the form of any one of dairy products, bread, confectionery, ice cream, beverage, tea, functional water and vitamin complex.

In addition, the health functional food composition may further include a food additive and compliance as a food additive is determined by the standards for the applicable item in accordance with General Regulations and General Test Methods of Korean Food Additives Codex approved by the Ministry of Food and Drug Safety, unless otherwise provided.

Examples of the items published in the above-mentioned “Korean Food Additives Codex” include chemical synthetics such as ketones, glycine, potassium citrate, nicotinic acid, and cinnamic acid and the like, natural additives such as persimmon color, licorice extract, crystalline cellulose, kaoliang color and guar gum and the like, mixed preparations such as L-sodiumglutamate preparation, alkaline agents for noodles, preservative formulation and a tar color formulation and the like.

At this time, the composition added to food including beverages in the process of manufacturing health functional foods can be appropriately added or subtracted as needed, and specifically, can be added to include 1 to 15% by weight based on 100 weight of the food.

The health functional food may provide nutritional benefits in addition to the therapeutic effect of the present invention, such as a nutritional supplement. In addition, the health functional food may be formulated to enhance the taste of the pharmaceutical composition and the health functional food composition according to the present invention, or to be more attractive for consumption because it is more similar to a general food item than the pharmaceutical composition.

Hereinafter, the present invention will be described in more detail through examples. These examples are only intended to illustrate the present invention in more detail, and it will be apparent to those skilled in the art that the scope of the present invention is not limited by these examples according to the gist of the present invention. The examples of the present invention are provided to more completely explain the present invention to those of ordinary skill in the art.

Tetragenococcus halophilus used in the following experimental examples was distributed from KCTC (#3720).

### [Experimental Example 1] Dendritic Cell Expression Analysis Experiment Using Flow Cytometry in Normal Mice and Behcet's Disease Mice

Cell expression frequencies of the dendritic cell activation molecules CD83 and CD40 were analyzed in normal and Behcet's disease mice. The results of comparing the control without any treatment on the two molecules and the case of treating the two molecules with T. Halophilus are as follows.

Table 1 below shows the results of the expression analysis of dendritic cells of the control group and Tetragenococcus halophilus in normal mice.

TABLE 1

| | Activating molecule | | | |
|---|---|---|---|---|
| | CD83 | | CD40 | |
| Treatment group | control | T. Halophilus | control | T. Halophilus |
| 1 | 32.4 | 24.4 | 33.9 | 21.2 |
| 2 | 38.5 | 27.0 | 34.8 | 18.6 |
| 3 | 40.2 | 26.8 | 29.8 | 19.9 |
| 4 | 37.7 | 28.0 | 35.9 | 23.6 |
| 5 | 38.1 | 27.4 | 33.3 | 26.4 |
| Average | 37.38 | 26.72 | 33.54 | 21.94 |
| SD | 2.94 | 1.38 | 2.30 | 3.10 |

| | | |
|---|---|---|
| P value | 0.00008 | 0.0001 |

Referring to Table 1, both the expression frequencies of CD83 and CD40 were decreased in the case of Tetragenococcus halophilus treatment. Through this, it can be confirmed that Tetragenococcus halophilus has an effect on the regulation of expression of dendritic cell activation molecules.

Table 2 below shows the results of the analysis of dendritic cell expression analysis of the control group and Tetragenococcus halophilus in Behcet's disease mice.

TABLE 2

| | Activating molecule | | | |
|---|---|---|---|---|
| | CD83 | | CD40 | |
| Treatment group | control | T. Halophilus | control | T. Halophilus |
| 1 | 41.0 | 42.9 | 23.3 | 33.9 |
| 2 | 46.6 | 46 | 24.1 | 29.6 |
| 3 | 52.0 | 27.4 | 20.4 | 33.8 |
| 4 | 40.6 | 19.9 | 32.6 | 31.4 |
| 5 | 34.5 | 16.6 | 42.1 | 17.2 |
| Average | 42.94 | 30.56 | 28.50 | 29.18 |
| SD | 6.63 | 13.31 | 8.85 | 6.93 |

| | | |
|---|---|---|
| P value | 0.09 | 0.89 |

Referring to Table 2, the frequency of expression of CD83 decreased in the case of Tetragenococcus halophilus treatment. Through this, it can be confirmed that Tetragenococcus halophilus has an effect on the regulation of expression of dendritic cell activation molecules.

### [Experimental Example 2] Behcet's Disease Mouse Experiment

Behcet's Disease-Like Mouse Model

ICR strain 5-week-old mice were inoculated with Herpes simplex virus type 1 onto the wounded ear auricle. Among the above mice, mice showing Behcet's disease characteristics accompanied by inflammatory symptoms such as vulvar inflammation, skin ulcers, and erythema were selected as a Behcet's disease-like mouse model and used in this experiment.

Method of Administration

Symptoms were confirmed after oral administration of 2×10⁸ Tetragenococcus halophilus once daily to a Behcet's disease-like mouse model for 10 days. As a control group, the symptoms were confirmed after administration of T. solitarius and T. koreensis belonging to the same genus thereof under the same conditions as Tetragenococcus halophilus, respectively.

Experiment Result

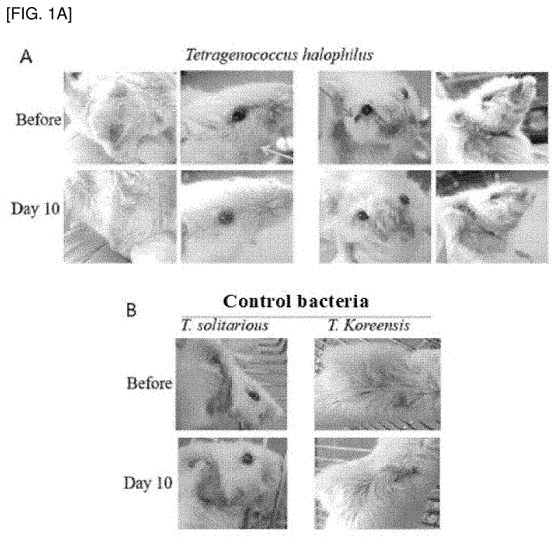

FIG. 1 shows the results according to an experimental example of the present invention.

Referring to FIG. 1, in a Behcet's disease-like mouse model, symptoms were taken before oral administration of Tetragenococcus halophilus for 10 days and as a result of comparing the improvement, the symptoms of vulvar inflammation, skin ulcer symptoms, and erythema symptoms were improved 10 days after administration of Tetragenococcus halophilus. On the other hand, it was confirmed that administration of T. solitarius and T. koreensis belonging to the same genus as a control group did not affect the improvement of symptoms. Through this, it can be confirmed that Tetragenococcus halophilus is specifically effective in improving symptoms of Behcet's disease or herpes simplex virus infection.

So far, specific examples of the present invention have been described. Those of ordinary skill in the art to which the present invention pertains will be able to understand that the present invention may be implemented in a modified form without departing from the essential characteristics of the present invention. Therefore, the disclosed embodiments should be considered from an illustrative point of view rather than a limiting point of view. The scope of the present invention is shown in the claims rather than the above description, and all differences within the scope equivalent thereto should be construed as being included in the present invention.

1. A method for treating Behcet's disease comprising administering a pharmaceutical composition comprising Tetragenococcus halophilus, a culture thereof or a lysate thereof to a subject in need thereof.

2. The method of claim 1, wherein the Tetragenococcus halophilus produces lactate, and the lactate is converted into butyrate in a body.

3. The method of claim 1, wherein the Tetragenococcus halophilus regulates expression of an activating molecule of dendritic cells.

4. The method of claim 1, wherein the composition further comprises a prebiotic compound, and the prebiotic compound is at least one selected from the group consisting of fructo-oligosaccharide (FOS), isomalto-oligosaccharide (IMO), xylo-oligosaccharide (XOS), chito-oligosaccharides (COS), pectin, inulin, β-glucan, polydextrose, D-tagatose, and acacia fibers.

5. A method for treating herpes simplex virus infection comprising administering a pharmaceutical composition comprising Tetragenococcus halophilus, a culture thereof or a lysate thereof to a subject in need thereof.

6. The method of claim 5, wherein the Tetragenococcus halophilus produces lactate, and the lactate is converted into butyrate in a body.

7. The method of claim 5, wherein the Tetragenococcus halophilus regulates expression of an activating molecule of dendritic cells.

8. The method of claim 5, wherein the composition further comprises a prebiotic compound, and the prebiotic compound is at least one selected from the group consisting of fructo-oligosaccharide (FOS), isomalto-oligosaccharide (IMO), xylo-oligosaccharide (XOS), chito-oligosaccharides (COS), pectin, inulin, β-glucan, polydextrose, D-tagatose, and acacia fibers.

9-10. (canceled)
